(19) United States
(12) Patent Application Publication
(10) Pub. No.: US 2012/0002166 A1
(43) Pub. Date: Jan. 5, 2012

(54) OPHTHALMOLOGIC APPARATUS AND OPHTHALMOLOGIC SYSTEM

(75) Inventors: Nobuhiro Tomatsu, Yokohama-shi (JP); Tomoyuki Makihira, Kamakura-shi (JP); Junko Nakajima, Tokyo (JP); Norihiko Utsunomiya, Machida-shi (JP)

(73) Assignee: CANON KABUSHIKI KAISHA, Tokyo (JP)

(21) Appl. No.: 13/166,977

(22) Filed: Jun. 23, 2011

(30) Foreign Application Priority Data
Jul. 5, 2010 (JP) 2010-152820
Jun. 17, 2011 (JP) 2011-135271

(51) Int. Cl. A61B 3/12; A61B 3/15; A61B 3/14

(52) U.S. Cl. 351/208; 351/206

(57) ABSTRACT

Provided are a fundus imaging apparatus and a fundus imaging method capable of alleviating loads on an operator and a patient in fundus imaging and of acquiring a high-resolution fundus image. The fundus imaging apparatus having a tracking function is configured to: determine whether or not a template is recorded based on specific information of an eye to be inspected (203); when the template is recorded, read out the template (204); execute template matching on an acquired fundus image (208); and execute tracking at the time of fundus imaging according to a result of the template matching (211).

### BACKGROUND OF THE INVENTION

1. Field of the Invention

The present invention relates to an ophthalmologic apparatus and an ophthalmologic system, and more particularly, to an ophthalmologic apparatus and an ophthalmologic system having a tracking function in a plane perpendicular to an eye axis of an eye to be inspected.

2. Description of the Related Art

In recent years, an ophthalmologic image typified by a fundus image is used in medical care as a medical image for disease follow-up or the like. For that purpose, various kinds of ophthalmologic equipment are used for taking the ophthalmologic image. For example, a fundus camera, an optical coherence tomography (hereinafter, referred to as OCT) apparatus, and a scanning laser ophthalmoscope (hereinafter, referred to as SLO) are used as optical equipment for taking a fundus image.

Such ophthalmologic equipment is used repeatedly on the same patient for the disease follow-up. Therefore, there have been proposed technologies involving recording an ophthalmologic image taken for the first time or conditions used in taking the ophthalmologic image and reading out the recorded ophthalmologic image or conditions when an ophthalmologic image is to be taken again, to thereby alleviate loads on an operator and a patient.

For example, Japanese Patent Application Laid-Open No. 2008-61847 discloses a technology involving recording a fundus image with identification information of a patient when the fundus image is taken, reading out the fundus image based on the identification information of the patient when a fundus image is to be taken again, and calculating a similarity to the newly taken fundus image. As represented by positions of blood vessels in a fundus, the fundus image shows a specific pattern for each individual. This pattern is utilized in matching with the patient information, to thereby prevent mistaking patients for each other due to an error of an operator.

Further, Japanese Patent Application Laid-Open No. H05-154108 discloses a technology involving, for the purpose of acquiring a fundus image at the same position as that of the previously taken image in disease follow-up, recording image taking conditions such as the position of a fixation lamp and the position of a focus lens, reading out the recorded image taking conditions when a new image is to be taken, and taking the new image with the read-out image taking conditions. This allows the image taking conditions similar to those used in the previous image taking may be set quickly, to thereby reduce the image taking time and alleviate loads on an operator and a patient.

Meanwhile, in recent years, the ophthalmologic equipment has increased in resolution for the purpose of detecting a smaller disease area. However, as the resolution increases, the effect of eye movement becomes unignorable. In particular, in the ophthalmologic equipment that scans the fundus to take a high-definition image, such as an OCT apparatus and an SLO, in taking one fundus image, when a fundus movement occurs during the image taking time, the acquired fundus image becomes discontinuous. Therefore, in order to reduce the effect of the eye movement and obtain a fundus image of high definition, an apparatus for detecting the eye movement has been attracting attention.

The eye movement may be measured by various methods such as the corneal reflection method (Purkinje image) and the search coil method. Among those methods, there has been studied a method of measuring the eye movement from a fundus image, which is simple and impose little burden on a subject.

In order to measure the eye movement from the fundus image, it is necessary to extract feature points from the fundus image. As the feature points of the fundus image, the macula, the optic nerve head, and the like may be used. However, in patients with an affected eye, the macula or the optic nerve head is often defective. Therefore, Japanese Patent Application Laid-Open No. 2001-070247 discloses a method involving selecting a candidate region from a fundus image, and detecting the presence or absence of a crossing of blood vessels in the candidate region on the conditions that four or more blood vessels pass the periphery of the candidate region and a blood vessel runs through the center of the candidate region.

As described above, it is generally desired for the ophthalmologic equipment to take a fundus image of high resolution while alleviating the burdens on the operator and the patient. However, a general technology for detecting and correcting a moving amount (hereinafter, referred to as tracking) involves extracting a feature point (hereinafter, referred to as template) as an index of the eye movement every time an image is taken and using the template for tracking. Therefore, time and tasks for extracting the template increase, which inevitably results in the burdens on the operator and the patient.

According to Japanese Patent Application Laid-Open No. 2008-61847, a fundus image is recorded with the patient information, to thereby read out the fundus image based on the patient information and calculate the similarity between images. The patient information and the fundus image are recorded in association with each other so that the burden on the operator may be reduced. Further, as to the similarity, there is disclosed a technology of superimposing the images based on the feature points. However, no technology for executing tracking in taking successive images of the fundus is disclosed, and the method does not allow calculating the eye movement consecutively at high speed.

According to Japanese Patent Application Laid-Open No. H05-154108, the image taking conditions used in taking an image for the first time are recorded with the patient information, and the conditions may be set based on the patient information the next time the fundus image is taken. Therefore, the burden on the operator may be alleviated, and the image may be taken easily. However, the detection of the fundus movement is not disclosed.

Further, in Japanese Patent Application Laid-Open No. 2001-070247, a small region in which the blood vessels cross is extracted, but there is no disclosure on recording the extracted small region as a template and reading out the recorded template when an image is to be taken again.

### SUMMARY OF THE INVENTION

The present invention has been made in view of the above-mentioned problems, and therefore has an object to provide an ophthalmologic apparatus and an ophthalmologic system capable of omitting or shortening extraction of a template in taking an image of a fundus so as to alleviate burdens on an operator and a patient when a fundus image of high definition is to be taken.

In order to solve the above-mentioned problems, the present invention provides an ophthalmologic apparatus and an ophthalmologic system configured as follows.

An ophthalmologic apparatus according to the present invention includes; a fundus imaging unit for acquiring a fundus image of an eye to be inspected, a template extracting unit for extracting a template from the acquired fundus image, a memory control unit for recording the extracted template and specific information identifying the eye to be inspected, of which the fundus image from which the template is extracted is acquired, in association with each other in a recording unit, a determination unit for determining whether or not a template associated with the specific information of the eye to be inspected, of which a fundus image is to be acquired by the fundus imaging unit, is recorded in the recording unit, a read-out unit for reading out, when it is determined that the associated template is recorded, the associated template from the recording unit; and a tracking unit for tracking the fundus image of the eye to be inspected by using the read-out template.

Further, an ophthalmologic system according to the present invention includes; a fundus imaging unit for acquiring a fundus image of an eye to be inspected, a template extracting unit for extracting a template from the acquired fundus image, a memory control unit for recording the extracted template and specific information identifying the eye to be inspected, of which the fundus image from which the template is extracted is acquired, in association with each other in a recording unit, a determination unit for determining whether or not a template associated with the specific information of the eye to be inspected, of which a fundus image is to be acquired by the fundus imaging unit, is recorded in the recording unit, a read-out unit for reading out, when it is determined that the associated template is recorded, the associated template from the recording unit; and a tracking unit for tracking the fundus image of the eye to be inspected by using the read-out template.

According to the present invention, it is possible to omit or shorten the extraction of a template in taking a fundus image so as to alleviate burdens on an operator and a patient when a fundus image of high definition is to be taken.

Further features of the present invention will become apparent from the following description of exemplary embodiments with reference to the attached drawings.

### BRIEF DESCRIPTION OF THE DRAWINGS

FIG. 1 is a diagram illustrating a configuration of a fundus imaging apparatus, which is an embodiment mode of an ophthalmologic apparatus according to a first embodiment of the present invention.

FIG. 2 is a flow chart illustrating an imaging method according to the first embodiment of the present invention.

FIG. 3 is a diagram illustrating a database according to the first embodiment of the present invention.

FIG. 4 is a diagram illustrating a fundus image according to the first embodiment of the present invention.

FIG. 5 is a flow chart illustrating an imaging method according to a second embodiment of the present invention.

FIG. 6 is a diagram illustrating template matching according to the first embodiment of the present invention.

FIG. 7 is a diagram illustrating a fundus image according to a third embodiment of the present invention.

FIGS. 8A and 8B are diagrams illustrating template extraction according to the third embodiment of the present invention.

FIGS. 9A and 9B are diagrams illustrating the template extraction according to the third embodiment of the present invention.

FIG. 10 is a diagram illustrating templates according to the first embodiment of the present invention.

FIGS. 11A and 11B are diagrams illustrating an eye movement according to the first embodiment of the present invention.

FIGS. 12A and 12B are diagrams illustrating another eye movement according to the first embodiment of the present invention.

FIGS. 13A and 13B are diagrams illustrating a still another eye movement according to the first embodiment of the present invention.

FIG. 14 is a diagram illustrating a disease area according to a fourth embodiment of the present invention.

FIG. 15 is a flow chart illustrating an imaging method according to the fourth embodiment of the present invention.

### DESCRIPTION OF THE EMBODIMENTS

Preferred embodiments of the present invention will now be described in detail in accordance with the accompanying drawings.

### First Embodiment

Hereinafter, an ophthalmologic apparatus according to an embodiment of the present invention is described in detail with reference to the accompanying drawings.

In this embodiment, the description is made on a scanning laser ophthalmoscope (SLO) to which the present invention is applied. Here, in particular, an apparatus for executing tracking by using a template recorded in advance is described.

(Scanning Laser Ophthalmoscope: SLO)

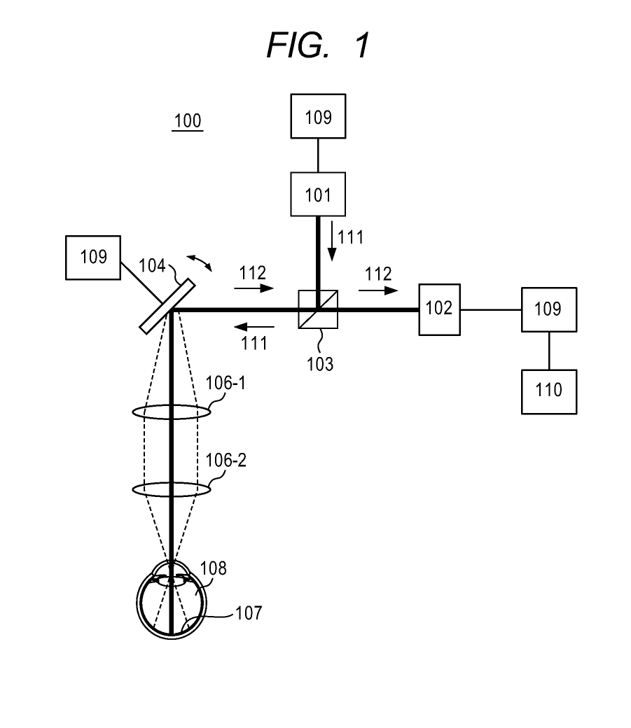

First, referring to FIG. 1, a schematic overall configuration of an optical system of the SLO according to this embodiment is described.

Note that, in FIG. 1, multiple boxes with the same reference numeral 109 representing a memory control and signal processing portion are illustrated for convenience, but those boxes are actually a single component.

An illumination beam 111 emitted from a light source 101 is deflected by a half mirror 103 and scanned by an XY scanner 104. Between the XY scanner 104 and an eye to be inspected 108, lenses 106-1 and 106-2 for illuminating a fundus 107 with the illumination beam 111 are arranged. For simplicity, the XY scanner 104 is illustrated as one mirror, but is actually composed of two mirrors, an X scanning mirror and a Y scanning mirror, disposed in proximity to each other. Therefore, the XY scanner 104 may raster scan the fundus 107 in a direction perpendicular to an optical axis.

After entering the eye to be inspected 108, the illumination beam 111 is reflected or scattered by the fundus 107 and then returned as a return beam 112. The return beam 112 enters the half mirror 103 again, and a beam transmitted therethrough enters a sensor 102. The sensor 102 converts a light intensity of the return beam 112 at each measurement point of the fundus 107 to a voltage, and feeds a signal indicating the voltage to the memory control and signal processing portion 109. The memory control and signal processing portion 109 uses the fed signal to generate a fundus image, which is a two-dimensional image. A part of the memory control and signal processing portion 109 cooperates with the optical system and the sensor 102 described above to constitute a fundus imaging unit for taking a fundus image in this embodiment. Further, the memory control and signal processing portion 109 extracts, from the two-dimensional image, a region of a predetermined shape and size that includes a feature point having a feature such as a crossing or branching area of blood vessels in the fundus as a template. Therefore, the template is image data of the region including the feature point. Here, the memory control and signal processing portion 109 has a function constituting a template extracting unit for executing the above-mentioned extraction of the template from the fundus image. Template matching is executed on a newly generated two-dimensional image by using the extracted template, to thereby calculate a moving amount of the eye to be inspected. Further, the memory control and signal processing portion 109 executes tracking depending on the calculated moving amount. In this embodiment, a method of executing the tracking in post-processing after taking the fundus image is described.

The memory control and signal processing portion 109 also includes a keyboard or mouse (not shown) and supports external input. Further, the memory control and signal processing portion 109 controls start and end of the fundus imaging. The memory control and signal processing portion 109 also includes a monitor (not shown) and may display the fundus image and specific information of the eye to be inspected. This allows an operator to observe the fundus in an image. Further, the template extracted by the memory control and signal processing portion 109 is recorded in a memory portion 110 with the input specific information of the eye to be inspected. Specifically, the extracted template and the specific information identifying the eye to be inspected, of which the fundus image from which the template is extracted is taken, are recorded in association with each other in the memory portion 110 under the memory control of the memory control and signal processing portion 109.

(Reading Out of Recorded Information)

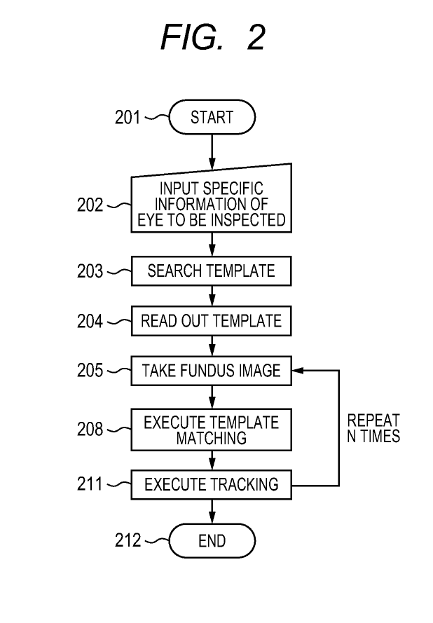

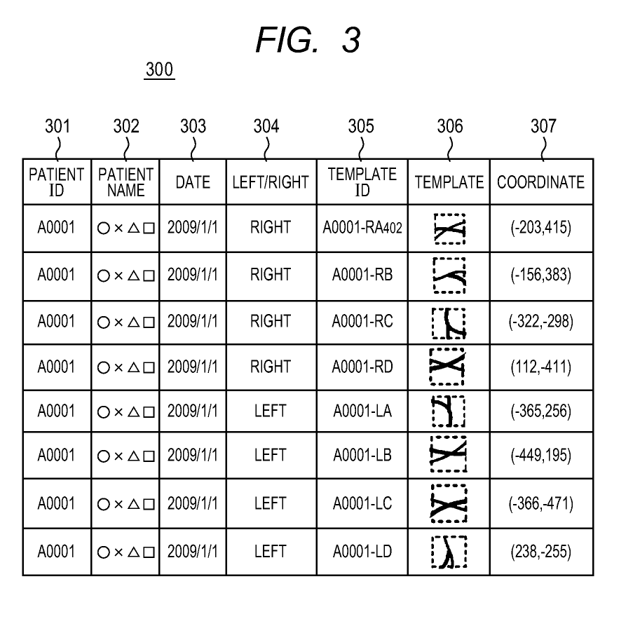

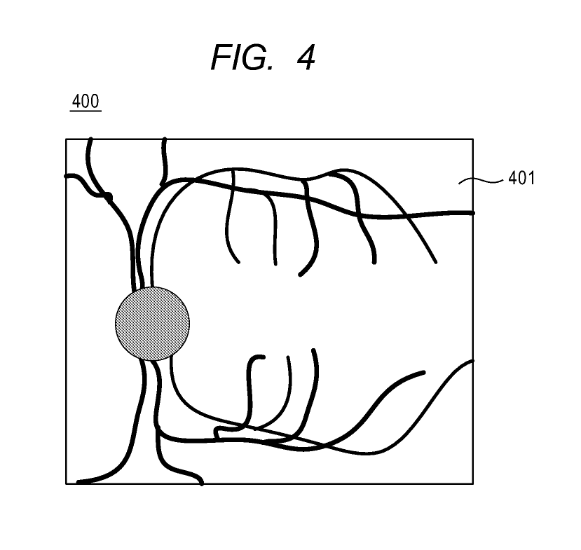

FIG. 2 illustrates a flow until tracking using a fundus imaging apparatus according to this embodiment. FIG. 3 illustrates recording the specific information of the eye to be inspected and the template. FIG. 4 illustrates the taken fundus image.

The operator, who is a user of the apparatus, inputs specific information of the eye to be inspected to the memory control and signal processing portion 109 or selects a record from multiple records of specific information of eyes to be inspected displayed in advance on the monitor of the memory control and signal processing portion 109 (Step 202). The memory control and signal processing portion 109 searches for a template recorded in the memory portion 110 based on the specific information of the eye to be inspected (Step 203). Here, the specific information of the eye to be inspected may at least contain information that allows identification of a patient and distinguishment between the left eye and the right eye of the patient. Through the above-mentioned processing, a determination function of the memory control and signal processing portion 109 determines whether a template associated with the specific information of the eye to be inspected, of which the image is taken, is recorded in the memory portion 110.

It is sufficient when one template is extracted for one eye to be inspected, but multiple templates are desirably extracted for more accurate tracking. In this embodiment, a case where four templates are extracted is described.

Further, information regarding fundus alignment may be added to the recorded templates. The fundus alignment as used herein means image taking conditions used when the fundus image is taken. The information regarding the fundus alignment is hereinafter referred to as fundus alignment information and includes various kinds of information to be set at the time of taking a fundus image. Examples of the fundus alignment information include information on the date on which the templates are extracted, position information of a fixation lamp, identification information indicating whether the eye to be inspected is the left eye or the right eye, coordinate information of the templates, position information of a focus lens, and the like. The fundus alignment information added here is useful in a case where images are taken over time of the same eye to be inspected under the same conditions. In particular, in disease follow-up, it is necessary to take images under substantially the same image taking conditions for comparison with the fundus images taken previously. Further, in a case where the template matching is executed using the templates recorded in the memory portion 110, as a fundus image to be newly taken is more similar to the fundus image from which the templates are extracted, there is a higher chance that a match is found. Therefore, the information on the fundus alignment for taking an image of the same eye to be inspected under the same conditions is desirably added to the recorded templates.

In this embodiment, the specific information of the eyes to be inspected and the templates are recorded in a database having a structure illustrated in FIG. 3. Each record in the database indicates a patient ID 301, a patient name 302, a date 303, left/right eye identification information 304, a template ID 305, a template 306, and template coordinates 307. When the operator inputs a patient ID or a patient name, the above-mentioned information is searched for a template. The left/right eye identification information 304 is information indicating whether the recorded template of the eye to be inspected is for the left eye or the right eye. When the template is recorded, the memory control and signal processing portion 109 reads out the template of interest from the memory portion 110 (Step 204). In the read-out processing, when the determination function determines that a template associated with the specific information of the eye to be inspected is recorded in the memory portion 110, a read-out function of the memory control and signal processing portion 109 reads out the associated template.

(Acquisition of Fundus Image)

After the template is read out, the memory control and signal processing portion 109 transmits a signal indicating the start of fundus imaging to the light source 101, the sensor 102, and the XY scanner 104, and starts taking the fundus image (Step 205).

The XY scanner 104 raster scans the fundus 107 with the illumination beam 111 emitted from the light source 101 in the direction perpendicular to the optical axis. The illumination beam 111 irradiating the fundus 107 is reflected or scattered by the fundus 107 and enters the sensor 102 as the return beam 112. The sensor 102 converts the light intensity of the return beam 112 at each measurement point of the fundus 107 to a voltage, and feeds a signal indicating the voltage to the memory control and signal processing portion 109. The memory control and signal processing portion 109 generates a two-dimensional image of the fundus 107 based on the fed signal.

(Template Matching)

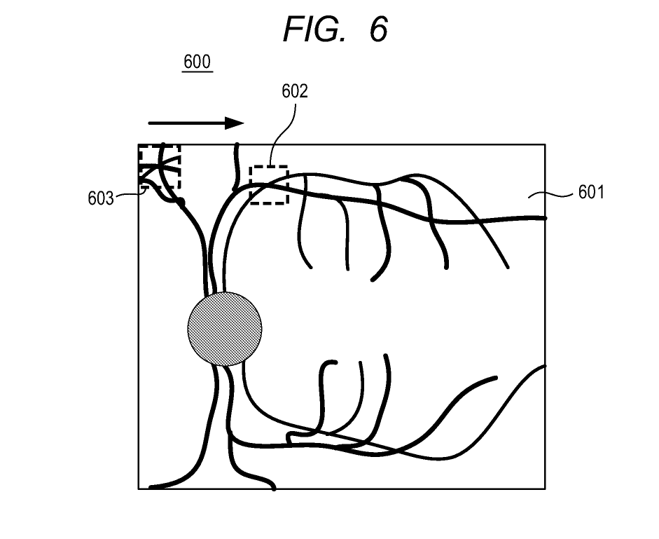

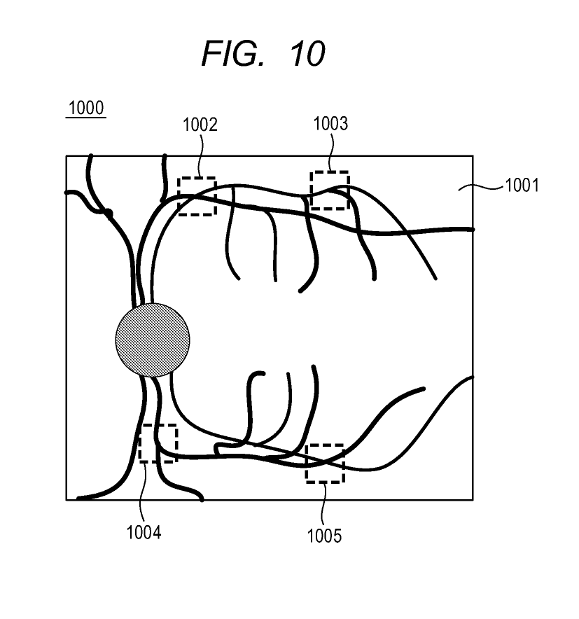

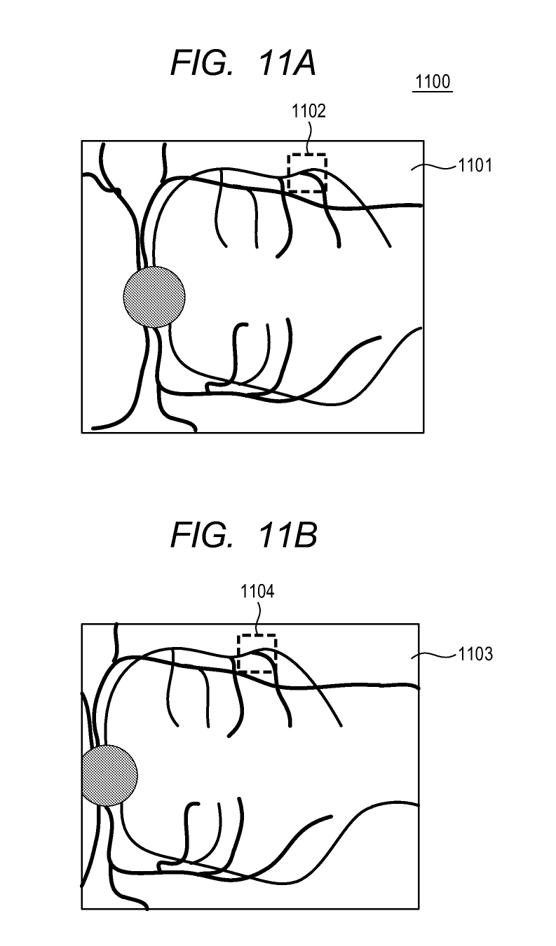

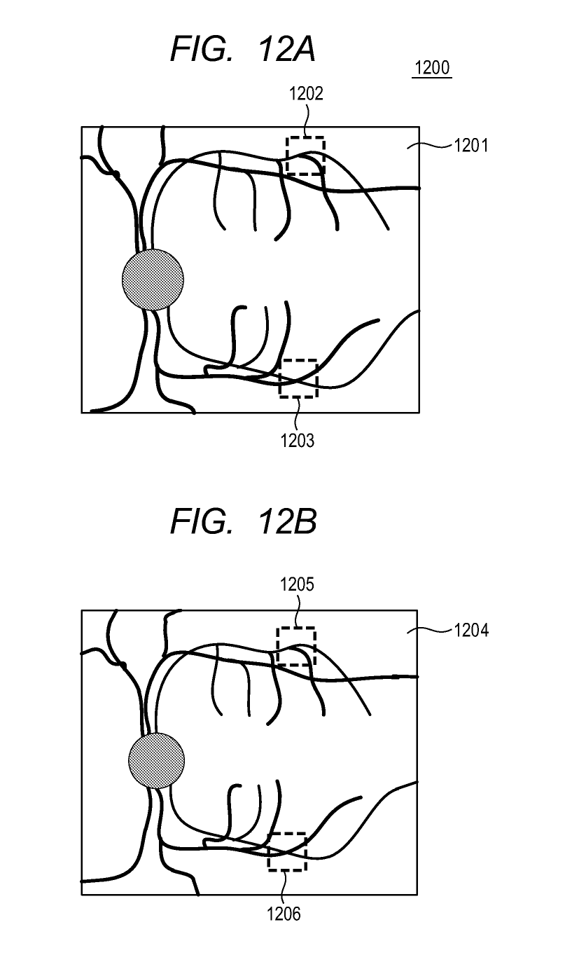

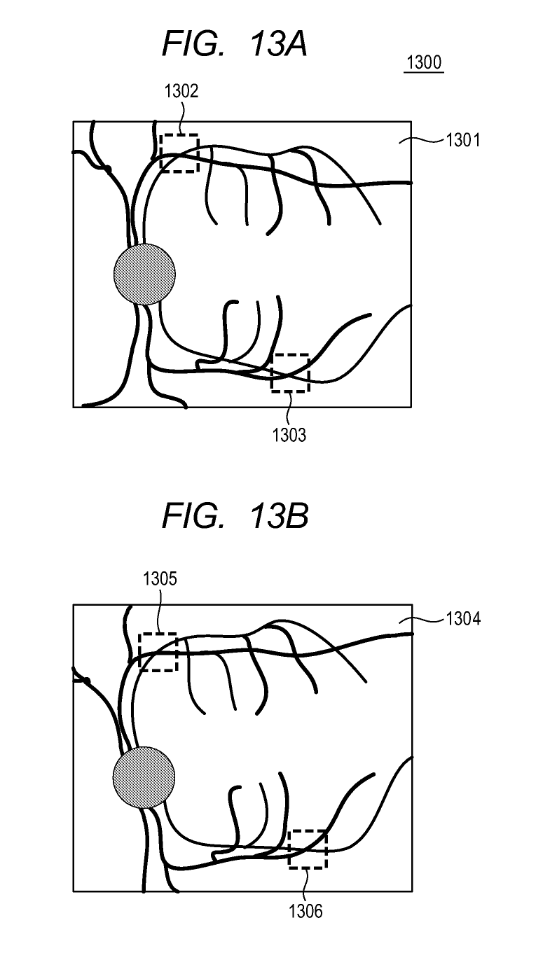

When one fundus image 400 is obtained, searching (hereinafter, referred to as matching) is executed on the acquired fundus image to find a region matching the read-out template (Step 208). As illustrated in FIG. 6, the matching is executed by shifting a template 603 over an acquired fundus image 601 and searching for a portion 602 matching the template 603. In this embodiment, as illustrated in FIG. 10, the template matching is executed using four templates 1002, 1003, 1004, and 1005. In this case, the tracking is possible as long as there is a match for at least one template. Therefore, when multiple templates are recorded, there is no need to find a match for all the recorded templates as long as there is a match for at least one template. Further, for improving the accuracy of tracking, reextraction of a template may be executed (details are provided in a third embodiment). For example, in a case where an eye movement in which feature points in fundus images move in parallel is to be detected, the eye movement may be detected by using at least one template (FIGS. 11A and 11B). Here, FIG. 11B illustrates a fundus image obtained as a result of the eye movement in which the feature points in a fundus image of FIG. 11A move in parallel. Further, in a case where an eye movement in which fundus images expand or contract is to be detected, the eye movement may be detected using at least two templates (FIGS. 12A and 12B). Here, FIG. 12B illustrates a fundus image obtained as a result of the eye movement in which a fundus image of FIG. 12A contracts. Further, in a case where an eye movement in which fundus images are rotated is to be detected, the eye movement may be detected by using at least two templates (FIGS. 13A and 138). Here, FIG. 13B illustrates a fundus image obtained as a result of the eye movement in which a fundus image of FIG. 13A is rotated. In this manner, the number of templates used for detecting the fundus movement may be set depending on the movement that the operator wants to detect, but it is desired that a match be found for a larger number of templates in order to detect the eye movement in more detail. Therefore, the case where four templates are associated with the specific information of the same eye to be inspected has been illustrated. However, the number of templates is not limited thereto, and it is preferred that any number of multiple templates be recorded at the same time.

(Tracking)

When a match is found for the templates, a tracking function for executing tracking of the fundus image of the eye to be inspected of the memory control and signal processing portion 109 executes the tracking of the fundus image (Step 211).

The first fundus image under the same image taking conditions has a template position with a shift amount of zero and is used as a reference image in the tracking. The second and subsequent fundus images are corrected by obtaining a shift amount with respect to the first image. Specifically, a mapping is obtained from Equation 1 based on the shift of each template, and coefficients a, b, c, d, e, and f are calculated by the least square method so that the difference between the obtained coordinates and the coordinates of the reference image is minimized.

$$( \begin{matrix}{X}^{′} \\ {Y}^{′}\end{matrix} ) = ( \begin{matrix}a & b \\ c & d\end{matrix} ) ( \begin{matrix}X \\ Y\end{matrix} ) + ( \begin{matrix}e \\ f\end{matrix} ) \qquad Equation 1$$

where X and Y are coordinates of a template in the reference image (first fundus image), and X′ and Y′ are coordinates of the template in the second or subsequent fundus image to be processed. The coefficients for correction obtained by Equation 1 may be used for correction of the second or subsequent fundus image to be processed in Equation 2, to thereby execute the tracking.

$$( \begin{matrix}X \\ Y\end{matrix} ) = {( \begin{matrix}a & b \\ c & d\end{matrix} )}^{- 1} \{ ( \begin{matrix}{X}^{′} \\ {Y}^{′}\end{matrix} ) - ( \begin{matrix}e \\ f\end{matrix} ) \} \qquad Equation 2$$

The above-mentioned processing is executed by the memory control and signal processing portion 109 to correct the acquired image, and the corrected fundus image is displayed on the monitor.

The processing from the taking of the fundus image (Step 205) to the tracking (Step 211) is repeated a desired number of times to take multiple fundus images. The taken images are displayed in succession on the monitor of the memory control and signal processing portion 109. At this time, the fundus image displayed on the monitor looks static to the operator because the tracking is executed.

By taking the fundus images as described above, when the specific information of the eye to be inspected and the templates are recorded, the template extraction is not executed and hence the template extraction is omitted or reduced. Therefore, the image taking time may be reduced, the burdens on the operator and the patient may be reduced, and a high resolution fundus image may be taken easily.

Note that, in this embodiment, the description is made only on the scanning laser ophthalmoscope (SLO). However, the method is also applicable to a so-called fundus camera for taking a fundus image, in particular, an optical coherence tomography (OCT) apparatus and other such apparatuses. Further, in this embodiment, the tracking in the post-processing is described. However, the method is also applicable to a case where real-time tracking using a scanner is executed.

### Second Embodiment

In a second embodiment of the present invention, referring to FIGS. 1, 3, and 5, a case where the templates are not recorded in the method described in the first embodiment is described.

Note that, in this embodiment, the configuration is similar to that in the first embodiment, and hence the description thereof is omitted.

(Reading Out of Recorded Information)

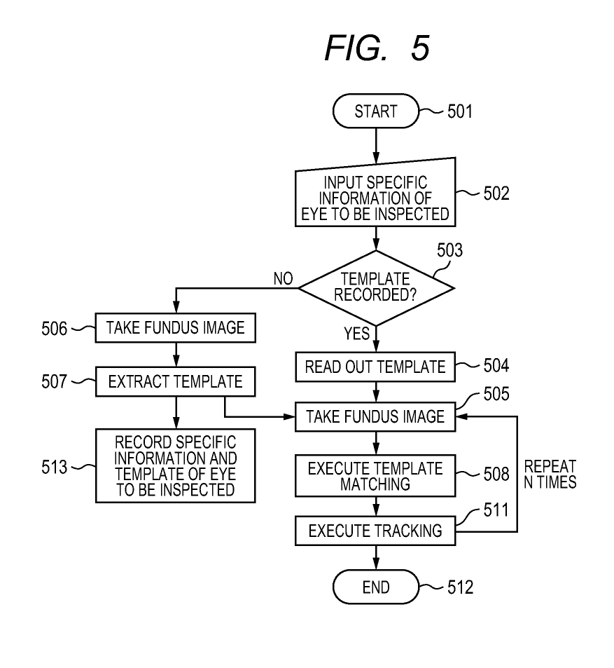

FIG. 5 illustrates a flow until tracking using a fundus observation apparatus according to this embodiment. Note that, the processing from the input or selection of the specific information of the eye to be inspected to the searching for a template in this embodiment (Steps 502 and 504) is similar to that in the first embodiment, and hence the description thereof is omitted.

(Template Extraction)

The operator inputs or selects the specific information of the eye to be inspected, and as a result of searching for a template, when the template is not recorded (Step 503; no), the memory control and signal processing portion 109 transmits a signal indicating the start of fundus imaging to the light source 101, the sensor 102, and the XY scanner 104. In response thereto, one fundus image for executing the template extraction is taken (Step 506).

The template extraction may be executed by using any method as long as the feature points such as branching and crossing of the blood vessels in the fundus image are extracted (Step 507). The extraction of the feature points are generally executed by the technology as described with reference to Japanese Patent Application Laid-Open No. 2001-070247, and hence the description thereof is omitted here.

(Recording of Templates)

The extracted templates are recorded with the specific information of the eye to be inspected, which is recorded in advance in the memory control and signal processing portion 109 or input by the operator, in the memory portion 110 (Step 513). The specific information of the eye to be inspected may at least contain information that allows identification of a patient and distinguishment between the left eye and the right eye of the patient. In this example, the patient ID 301, the patient name 302, the date 303, and the left/right eye identification information 304 are recorded as the specific information of the eye to be inspected. Further, the template IDs 305 as well as the image data 306 and the template coordinates 307 are added to the templates to be recorded with the specific information of the eye to be inspected (see FIG. 3).

(Acquisition of Fundus Image)

After completion of the template extraction, the memory control and signal processing portion 109 transmits a signal indicating the start of fundus imaging to the light source 101, the sensor 102, and the XY scanner 104, and starts taking the fundus image (Step 505).

Note that, the acquisition of the fundus image in this embodiment is similar to that in the first embodiment, and hence the description thereof is omitted.

(Template Matching)

After the fundus imaging is executed and a new fundus image is acquired, searching (hereinafter, referred to as matching) is executed on the newly acquired fundus image to find regions matching the read-out templates (Step 508). The extraction of the new templates is executed by a template extraction function of the memory control and signal processing portion 109 based on the determination by the determination function of the memory control and signal processing portion 109 that there is no template associated with the specific information of the eye to be inspected.

Note that, the template matching in this embodiment is similar to that in the first embodiment, and hence the description thereof is omitted.

(Tracking)

When a match is found for the templates in the taken fundus image, tracking is executed (Step 511).

Note that, the tracking in this embodiment is similar to that in the first embodiment, and hence the description thereof is omitted here.

By taking the fundus image as described above, even when the templates are not recorded, the templates are extracted from the new fundus image. Therefore, the burden on the operator may be reduced, and the fundus image may be taken easily. Further, when the templates are not recorded, template registration processing is simplified to reduce the burden on the operator, and further the burden on the patient may be reduced the next time an image is taken.

Note that, in this embodiment, the description is made on the SLO. However, the method is also applicable to a so-called fundus camera for taking a fundus image, in particular, an OCT apparatus and other such apparatuses. Further, in this embodiment, the tracking in the post-processing is described. However, the method is also applicable to a case where real-time tracking using a scanner is executed.

### Third Embodiment

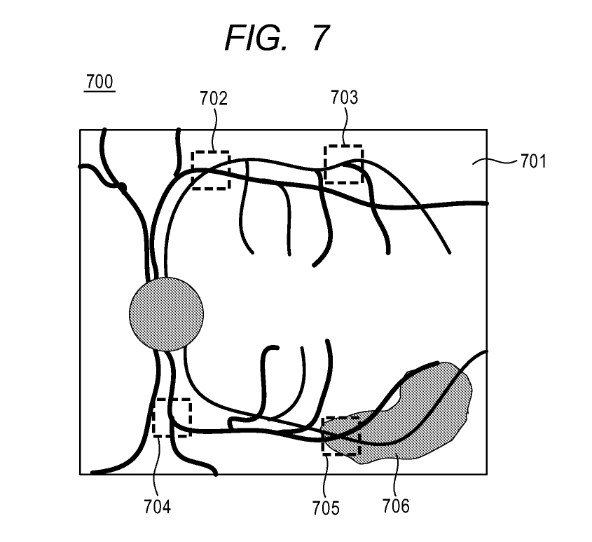

In the third embodiment of the present invention, referring to FIGS. 7, 8A and 8B, and 9A and 9B, for a case where there is a template for which no match is found in the first taken fundus image in the processing described in the first embodiment, a method involving reacquiring a template and then executing the tracking is described.

As illustrated in FIG. 7, when the difference in contrast between the blood vessels and other portions is small due to the effect of a disease 706 or the like in a portion 705 that should match a recorded template, the template matching cannot be executed. Therefore, a new template is set in a portion different from the recorded template to execute tracking.

Note that, in this embodiment, the configuration is similar to that in the first embodiment, and hence the description thereof is omitted. Further, the same description as in the first and second embodiments is omitted.

(Template Reextraction)

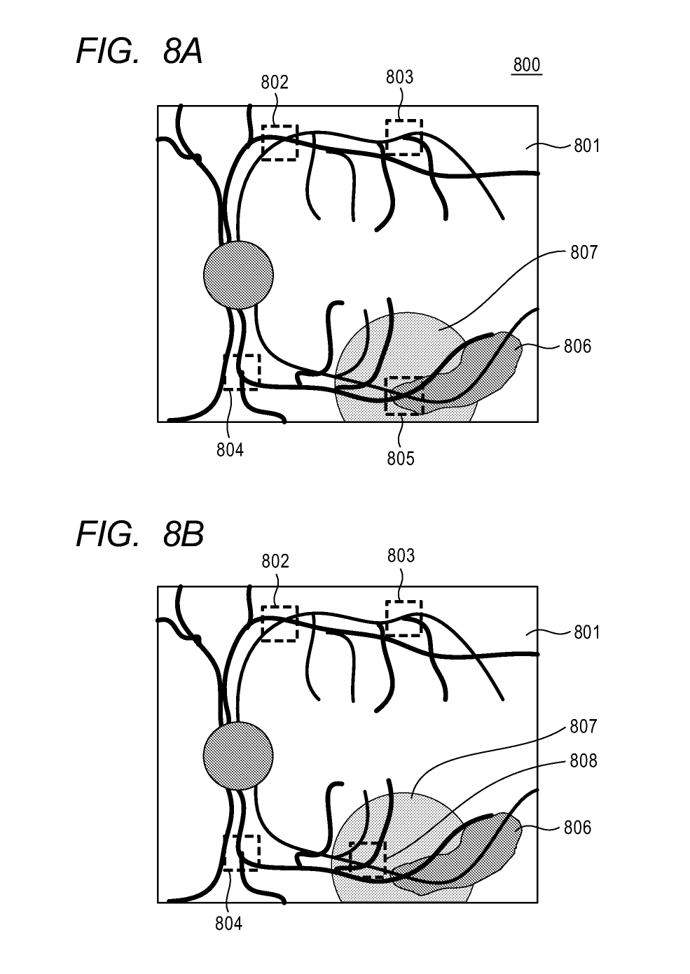

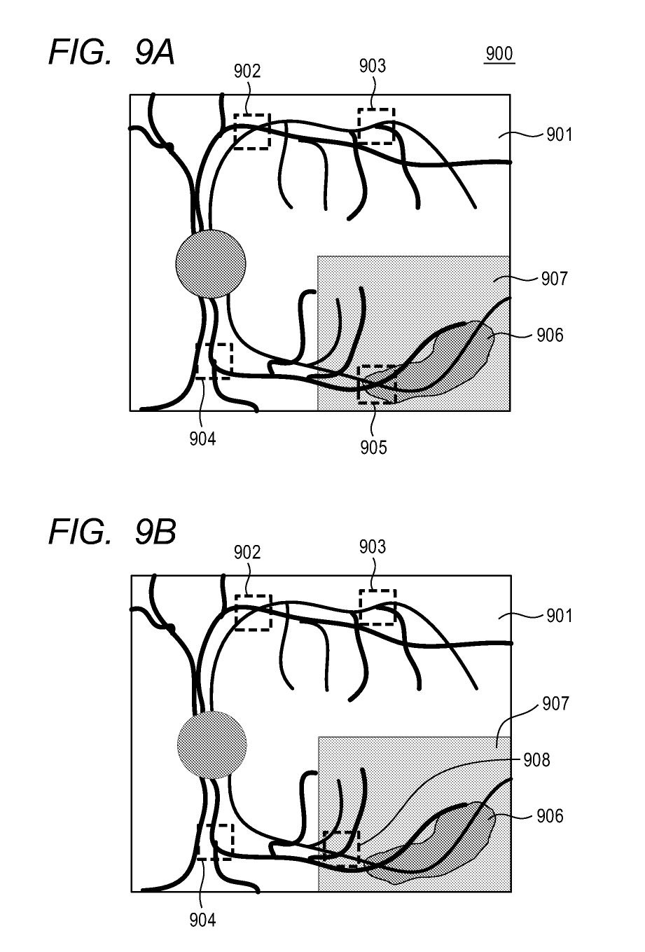

In the template matching using the read-out templates, when there is a template for which no match is found, reextraction is executed for the template for which no match is found in order to increase the tracking accuracy and to acquire an image of higher definition. The template for which no match is found is considered unsuitable for use and is not used for the subsequent processing. The reextraction is executed only for the template for which no match is found. The template may be reextracted by searching the entire fundus image for a feature point. However, it is sufficient when searching for a feature point is restricted to a region neighboring the template for which no match is found, to thereby reextract the template. The search area at this time is a concentric region 807 around the coordinates of the template for which no match is found as illustrated in FIG. 8A, and a new template 808 including a suitable feature point in the concentric region 807 may be extracted (see FIG. 8B). Alternatively, as illustrated in FIG. 9A, the same quadrant 907 in the fundus image as a quadrant including the template for which no match is found is set, and a new template including a suitable feature point in the quadrant 907 may be extracted as illustrated in FIG. 9B. Note that, it is preferred that the range of the concentric region 807 or the same quadrant 907 be set as a predetermined region obtained by multiplying the size of the template or the angle of view of the displayed fundus image as a reference by a predetermined coefficient. At this time, when a template for which a match is found is located in the concentric region 807, the template may be extracted redundantly. To address this problem, the region of the template for which a match is found may be excluded from the search area. In other words, it is preferred that, when there is a template for which a match is found, the region for extracting a new template be the original template extraction region excluding the template for which a match is found. In this manner, the template extraction is executed by restricting the search area for the template. Regarding the template extraction of this embodiment, the technology as described with reference to Japanese Patent Application Laid-Open No. 2001-070247 is publicly known, and hence the description thereof is omitted here.

(Template Matching)

The memory control and signal processing portion 109 executes the template matching on sequentially acquired fundus images by using the templates for which a match is found and the reextracted template.

The tracking in this embodiment is similar to that in the first embodiment, and hence the description thereof is omitted.

By reextracting the template for taking the fundus images as described above, the fundus images may be acquired with higher tracking accuracy. Further, the search area is restricted so that the time needed for the template extraction may be reduced. Note that, in this embodiment, the description is made on the SLO. However, the method is also applicable to a so-called fundus camera for taking a fundus image, in particular, an OCT apparatus and other such apparatuses. Further, in this embodiment, the tracking in the post-processing is described. However, the method is also applicable to a case where real-time tracking using a scanner is executed.

### Fourth Embodiment

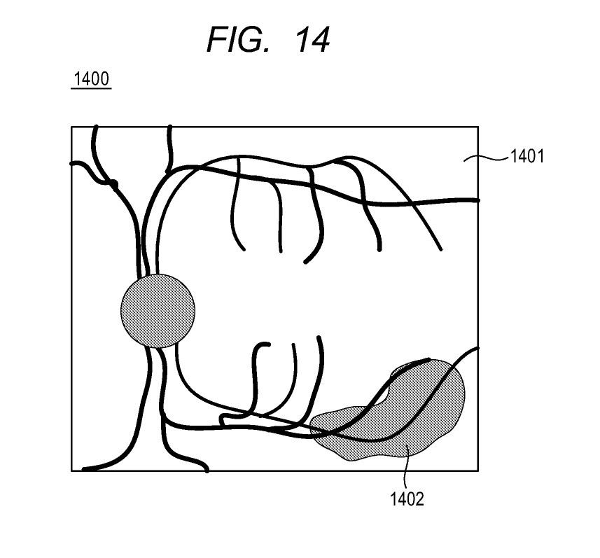

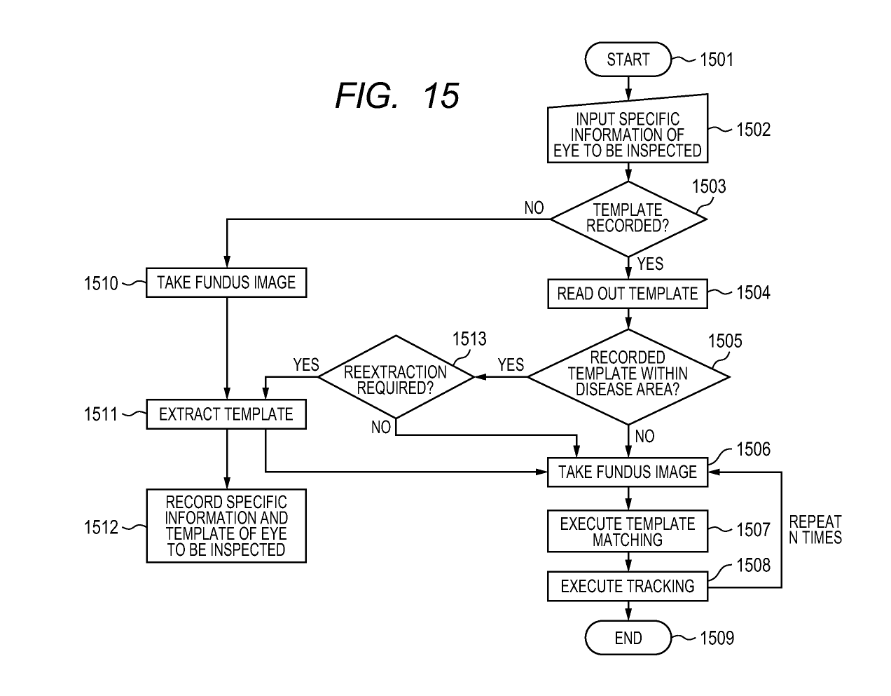

In a fourth embodiment, referring to FIGS. 14 and 15, a method of reacquiring a template based on disease information in the processing described in the first embodiment is described.

Note that, in this embodiment, the configuration is similar to that in the first embodiment, and hence the description thereof is omitted. Further, the same description as in the first, second, and third embodiments is omitted.

(Information Recording)

When the fundus is affected with a disease and the disease portion is known in advance, the operator inputs the disease information to the memory control and signal processing portion 109. The disease information to be input in this example includes, for example, a disease name, a disease area, a range, and the recording date, and is recorded in association with the specific information of the eye to be inspected. Also when a treatment or operation is performed on the affected eye, the operator inputs, for example, the details of the treatment, the course of treatment, the details of the operation, fundus information after the operation, and the like to be recorded by the memory control and signal processing portion 109. In this case, the disease area is specified by the operator selecting the disease area on the fundus image taken in the past (1402 in FIG. 14). The input information on the disease area is converted to coordinate information in the fundus image.

(Determination on Use of Templates)

Based on the specific information of the eye to be inspected, the templates are read out from the memory control and signal processing portion 109, and at the same time, the template coordinates recorded in association with the templates are also read out (1504 in FIG. 15). The thus read-out coordinates are compared with the information on the disease area and the range recorded in advance by the operator in the memory control and signal processing portion 109, and it is determined whether the template coordinates are within the disease area (1505 in FIG. 15). Therefore, the memory control and signal processing portion 109 further records at least one of the disease information, operation information, and treatment information, and a region functioning as a decision unit in the memory control and signal processing portion 109 decides whether or not to use a template based on any one of the disease information, the operation information, or the treatment information. When it is determined that the template is not in the disease area, the template read out from the memory control and signal processing portion 109 is used for tracking. When it is determined that the template is in the disease area, on the other hand, the template recorded in the memory control and signal processing portion 109 is not used for tracking.

Criteria used in determining whether or not to reextract the templates may be decided arbitrarily by the operator (1513 in FIG. 15). The determination criteria may include, for example, how much the template is affected by the disease area, how old is the image from which the recorded template is extracted, and the like. For example, it is decided whether or not to use a template based on the above-mentioned information on the date on which the template is extracted. Those determination criteria are input in advance by the operator to the memory control and signal processing portion 109, and the determination is made by the memory control and signal processing portion 109 at the time of taking the fundus image. However, the present invention is not limited thereto, and the operator may arbitrarily decide on the determination criteria depending on what to examine. In addition to the disease information, the operation information, the treatment information, and the like, region information may be included in advance as information used in deciding whether or not to use the recorded template.

(Template Reextraction)

A necessary number of templates are reextracted as needed (1511 in FIG. 15). When it is determined that the reextraction of the templates is not required, the reextraction of the templates is not necessarily needed. The details are similar as in the third embodiment (template reextraction), and hence the description thereof is omitted.

(Template Matching)

The memory control and signal processing portion 109 executes the template matching on sequentially acquired fundus images by using the templates for which a match is found and the reextracted template.

The tracking in this embodiment is similar to that in the first embodiment, and hence the description thereof is omitted.

By using the disease information recorded in advance as described above, the template for which no match is found may be eliminated in advance, and hence matching failure does not occur and the fundus image may be taken with higher tracking accuracy. Note that, in this embodiment, the description is made on the SLO. However, the method is also applicable to a so-called fundus camera for taking a fundus image, in particular, an OCT apparatus and other such apparatuses. Further, in this embodiment, the tracking in the post-processing is described. However, the method is also applicable to a case where real-time tracking using a scanner is executed.

### Another Embodiment

Further, the present invention is realized by executing the following processing. That is, in the processing, software (program) for implementing the functions of the above-mentioned embodiments is supplied to a system or an apparatus via a network or various recording mediums and is read out and executed by a computer (or CPU, MPU, or the like) of the system or the apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-152820, filed Jul. 5, 2010, and Japanese Patent Application No. 2011-135271, filed Jun. 17, 2011 which are hereby incorporated by reference herein in their entirety.

What is claimed is:

1. An ophthalmologic apparatus, comprising:
a fundus imaging unit for acquiring a fundus image of an eye to be inspected;
a template extracting unit for extracting a template from the acquired fundus image;
a memory control unit for recording the extracted template and specific information identifying the eye to be inspected, of which the fundus image from which the template is extracted is acquired, in association with each other in a recording unit;
a determination unit for determining whether or not template associated with the specific information of the eye to be inspected, of which a fundus image is to be acquired by the fundus imaging unit, is recorded in the recording unit;
a read-out unit for reading out, when it is determined that the associated template is recorded, the associated template from the recording unit; and
a tracking unit for tracking the fundus image of the eye to be inspected by using the read-out template.

2. An ophthalmologic apparatus according to claim 1, wherein, when it is determined that the associated template is not recorded, the template extracting unit extracts a new template.

3. An ophthalmologic apparatus according to claim 1, wherein the specific information of the eye to be inspected contains at least one of a patient ID and information indicating whether the eye to be inspected is a left eye or a right eye.

4. An ophthalmologic apparatus according to claim 1, wherein the template comprises multiple pieces of image data of crossing or branching areas of blood vessels in a fundus of the eye to be inspected.

5. An ophthalmologic apparatus according to claim 1, wherein the fundus imaging unit comprises any one of a fundus camera, an optical coherence tomography (OCT) apparatus, and a scanning laser ophthalmoscope (SLO).

6. An ophthalmologic apparatus according to claim 1, wherein the recording unit records information regarding fundus alignment, which is an image acquisition condition of the fundus image from which the template is extracted, in association with the template and the specific information of the eye to be inspected.

7. An ophthalmologic apparatus according to claim 6, wherein the information regarding fundus alignment contains at least one of information on a date on which the template is extracted, coordinate information of a fixation lamp, information indicating whether the eye to be inspected is a left eye or a right eye, and position information of a focus lens.

8. An ophthalmologic apparatus according to claim 1, wherein, when the recording unit records multiple templates and there is a template for which no match is found in a newly acquired fundus image, the template for which no match is found is prevented from being used.

9. An ophthalmologic apparatus according to claim 1, wherein, when the recording unit records multiple templates and there is a template for which no match is found in a newly acquired fundus image, a new template is extracted in a predetermined region in the newly acquired fundus image that includes the template for which no match is found.

10. An ophthalmologic apparatus according to claim 9, wherein the predetermined region is one of a region defined by a concentric circle centered at coordinates of the template for which no match is found, and the same quadrant in the newly acquired fundus image as a quadrant including the coordinates of the template for which no match is found.

11. An ophthalmologic apparatus according to claim 10, wherein, when the region defined by the concentric circle includes a template which is recorded in the recording unit and for which a match is found, the region excluding a region of the template for which a match is found is set as a region for extracting a template.

12. An ophthalmologic apparatus according to claim 1, further comprising a decision unit for deciding whether or not to use a template based on information on a date on which the template is extracted.

13. An ophthalmologic apparatus according to claim 1, further comprising a decision unit,
wherein the recording unit further records at least one of disease information, operation information, and treatment information, and
wherein the decision unit decides whether or not to use a template based further on any one of the disease information, the operation information, and the treatment information.

14. An ophthalmologic apparatus according to claim 13, wherein the disease information, the operation information, and the treatment information contain region information for deciding whether or not to use the recorded template.

15. An ophthalmologic system, comprising:
a fundus imaging unit for acquiring a fundus image of an eye to be inspected;
a template extracting unit for extracting a template from the acquired fundus image;
a memory control unit for recording the extracted template and specific information identifying the eye to be inspected, of which the fundus image from which the template is extracted is acquired, in association with each other in a recording unit;
a determination unit for determining whether or not a template associated with the specific information of the eye to be inspected, of which a fundus image is to be acquired by the fundus imaging unit, is recorded in the recording unit;
a read-out unit for reading out, when it is determined that the associated template is recorded, the associated template from the recording unit; and
a tracking unit for tracking the fundus image of the eye to be inspected by using the read-out template.

16. A recording medium having a program stored therein, the program causing a computer to execute functions of the ophthalmologic system according to claim 15.
